(19) United States
(12) Patent Application Publication
(10) Pub. No.: US 2021/0389582 A1
(43) Pub. Date: Dec. 16, 2021

(54) ENDOSCOPE MOTHERBOARD, ENDOSCOPE AND DETECTION METHOD

(71) Applicant: AUTEL INTELLIGENT TECHNOLOGY CORP., LTD., Shenzhen (CN)

(72) Inventor: Weilin WANG, Shenzhen (CN)

(21) Appl. No.: 17/446,232

(22) Filed: Aug. 27, 2021

Related U.S. Application Data
(63) Continuation of application No. PCT/CN2020/080414, filed on Mar. 20, 2020.

(30) Foreign Application Priority Data
Mar. 21, 2019 (CN) 201910218485.4

(51) Int. Cl. G02B 23/24; H04N 5/225; H04L 12/26; H04W 84/12

(52) U.S. Cl. CPC G02B 23/2484; H04W 84/12; H04L 43/0811; H04N 5/2257

(57) ABSTRACT

An endoscope motherboard, an endoscope and a detection method are provided. The endoscope motherboard is connected to a terminal and a camera module. The endoscope motherboard includes a communication interface, a wireless communication module and an adapter board. The communication interface and the wireless communication module are connected to the adapter board. The adapter board is connected to the camera module. The communication interface or the wireless communication module is connected to the terminal. The adapter board includes a detection module and a gating switch. The detection module detects a status of connection between the communication interface or the wireless communication module and the terminal and transmit a signal to the gating switch according to the status of connection. The gating switch connects the camera module to the communication interface or the wireless communication module according to the signal.

### CROSS-REFERENCE TO RELATED APPLICATIONS

The present application is a continuation application of International Application No. PCT/CN2020/080414, filed on Mar. 20, 2020, which claims priority of Chinese Patent Application No. 201910218485.4, filed on Mar. 21, 2019, which is incorporated herein by reference in its entirety.

### TECHNICAL FILED

The disclosure relates to the technical field of endoscopes, and in particular, to an endoscope motherboard, an endoscope and a detection method.

### BACKGROUND

An endoscope is a device configured to observe areas that cannot be directly observed by human eyes. For example, the endoscope is applicable to maintenance and repair of some large-scale devices in the industrial field, or is applicable to otolaryngological inspection in the medical field. The endoscope is mainly composed of a camera, a probe tube, a control module and a display module. The probe tube is connected to the camera and the control module to transmit an image acquired by the camera to the control module and display the image by using the display module.

With the popularization of smart terminals, the endoscope is combined with a smart terminal (such as a smart phone). The endoscope communicates with the terminal by using only a USB cable, or communicates with the terminal by using only a Wi-Fi module. Conventional endoscopes cannot support the USB communication and the Wi-Fi communication at the same time.

### SUMMARY OF THE DISCLOSURE

Embodiments of the disclosure provide an endoscope motherboard, an endoscope and a detection method, which can support both wired communication and wireless communication.

In the embodiments of the disclosure, the following technical solutions are disclosed:

In a first aspect, an embodiment of the disclosure provides an endoscope motherboard configured to be connected to a terminal and a camera module. The endoscope motherboard includes a communication interface, a wireless communication module and an adapter board. The communication interface and the wireless communication module are connected to the adapter board. The adapter board is connected to the camera module. The communication interface or the wireless communication module is configured to be connected to the terminal. The adapter board includes a detection module and a gating switch. The detection module is configured to detect a status of connection between the communication interface or the wireless communication module or a status of connection between the wireless communication module and the terminal, and the terminal and transmit a signal to the gating switch according to the status of connection. The gating switch is configured to connect the camera module to the communication interface or the wireless communication module according to the signal.

The communication interface is connected to the detection module and the gating switch. When the communication interface is connected to the terminal, the detection module is configured to transmit a first signal to the gating switch, so that the gating switch connects the camera module to the communication interface. When the communication interface is not connected to the terminal, the detection module is configured to transmit a second signal to the gating switch, so that the gating switch connects the camera module to the wireless communication module.

When the communication interface is not connected to the terminal, the detection module is configured to detect whether a signal from the wireless communication module is received, and if the signal from the wireless communication module is received, transmit the second signal to the gating switch.

When the communication interface is connected to the terminal, the signal transmission port is configured to transmit an image signal to the terminal, and/or transmit a control signal to the camera module, so that the camera module performs photographing according to the control signal. The image signal is transmitted by the camera module and the control signal is transmitted by the terminal.

The communication interface further includes a power supply port. When the communication interface is connected to the terminal, the terminal supplies power to the camera module through the power supply port.

The endoscope motherboard further includes an earphone interface and a photographing button connected to the earphone interface. The earphone interface is configured to be connected to the terminal, and transmit a trigger signal to the terminal, so that the terminal transmits the control signal according to the trigger signal. The trigger signal is transmitted by the photographing button.

The wireless communication module includes a controller connected to the gating switch. When the wireless communication module is connected to the terminal, the controller is configured to receive the image signal transmitted by the camera module and wirelessly transmit the image signal to the terminal, and/or wirelessly receive the control signal transmitted by the terminal, so that the camera module performs photographing according to the control signal.

The photographing button is further connected to the controller. When the wireless communication module is connected to the terminal, the controller is further configured to receive the trigger signal transmitted by the photographing button and wirelessly transmit the trigger signal to the terminal, so that the terminal transmits the control signal according to the trigger signal.

The wireless communication module further includes a power supply connected to the gating switch. When the wireless communication module is connected to the terminal, the power supply supplies power to the camera module.

The endoscope motherboard further includes a power supply control switch. One end of the power supply control switch is connected to the gating switch. Another end of the power supply control switch is connected to a flash light of the camera module to control a brightness of the flash light of the camera module.

The wireless communication module is one or more of a radio frequency signal communication module, an infrared signal communication module and a Wi-Fi signal communication module.

In a second aspect, an embodiment of the disclosure provides an endoscope, including a camera module and the endoscope motherboard described above.

In a third aspect, an embodiment of the disclosure provides a detection method applicable to an endoscope motherboard. The endoscope motherboard includes a communication interface, a wireless communication module and an adapter board. The communication interface and the wireless communication module are connected to the adapter board. The adapter board is connected to the camera module. The communication interface or the wireless communication module is configured to be connected to the terminal. The adapter board includes a detection module and a gating switch. The detection method includes: detecting, by the detection module, a status of connection between the communication interface and the terminal or a status of connection between the wireless communication module and the terminal, and transmitting a signal to the gating switch according to the status of connection; and connecting, by the gating switch, the camera module to the communication interface or the wireless communication module according to the signal.

According to the endoscope motherboard in the embodiments of the disclosure, the communication interface and the wireless communication module are connected to the adapter board and the adapter board is connected to the camera module. The adapter board includes the detection module and the gating switch. The detection module detects the status of connection between the communication interface and the terminal or a status of connection between the wireless communication module and the terminal, and transmits the signal to the gating switch according to the status of connection. The gating switch connects the camera module to the communication interface or the wireless communication module according to the signal transmitted by the detection module. In this way, the endoscope motherboard can support both wired communication and wireless communication. Therefore, a user may freely select the wired communication or the wireless communication, improving user experience.

### BRIEF DESCRIPTION OF THE DRAWINGS

One or more embodiments are exemplarily described with reference to the corresponding figures in the accompanying drawings. The descriptions are not to be construed as limiting the embodiments. Elements in the accompanying drawings that have same reference numerals are represented as similar elements. Unless otherwise particularly stated, the figures in the accompanying drawings are not drawn to scale.

FIG. 1 is a schematic structural diagram of a detection system according to an embodiment of the disclosure.

FIG. 2 is a schematic structural diagram of an endoscope motherboard of a detection system in FIG. 1.

FIG. 3 is a schematic structural diagram of the detection system in FIG. 1.

FIG. 4 is a schematic structural diagram of an adapter board of the endoscope motherboard in FIG. 2.

FIG. 5 is a schematic flowchart of a detection method according to an embodiment of the disclosure.

### DETAILED DESCRIPTION OF THE DISCLOSURE

The technical solutions in the embodiments of the disclosure are described below in detail with reference to the accompanying drawings in the embodiments of the disclosure. Apparently, the described embodiments are some rather than all of the embodiments of the disclosure. All other embodiments obtained by those of ordinary skill in the art based on the embodiments of the disclosure without any inventive effort shall fall within the protection scope of the disclosure.

In addition, technical features involved in implementations of the disclosure that are described below may be combined with each other provided that no conflict occurs.

The embodiments of the disclosure provide an endoscope motherboard, an endoscope and a detection method, which can support both wired communication and wireless communication.

The endoscope motherboard in the embodiments of the disclosure may be connected to a terminal and a camera module to perform the detection method in the embodiments of the disclosure.

Specifically, the following further describes the embodiments of the disclosure with reference to the accompanying drawings.

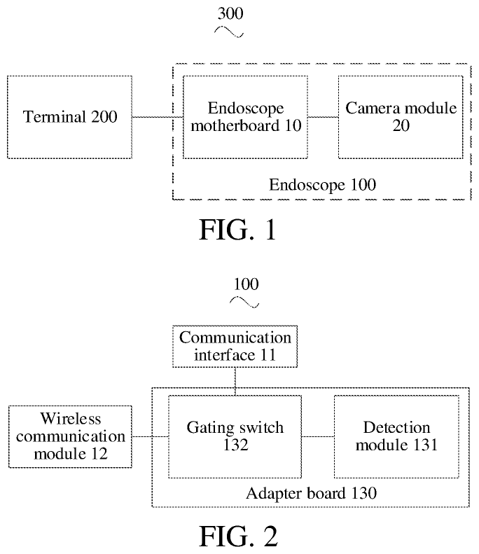

Referring to FIG. 1, FIG. 1 is a schematic structural diagram of a detection system according to an embodiment of the disclosure. As shown in FIG. 1, the detection system 300 includes an endoscope 100 and a terminal 200. The endoscope 100 and the terminal 200 are connected.

The terminal 200 may exist in a plurality of forms, including but is not limited to the following: (1). Mobile communication device: which has a mobile communication function and is mainly intended to provide voice and data communication. Such type of terminals include smart phones, functional phones and the like. (2). Ultra-mobile personal computer device: which belongs to a personal computer category. The personal computer device has computing and processing functions and generally offers mobile Internet access. Such type of terminals include a PDA, an MID, a UMPC device and the like. (3). Portable entertainment device: which is capable of displaying and playing content. Such type of devices include an audio and video player, a handheld game console, an e-book, a smart toy and the like. (4). Other electronic apparatuses having a data interaction function.

The endoscope 100 includes an endoscope motherboard 10 and a camera module 20. The endoscope motherboard 10 is connected to the camera module 20. The endoscope motherboard 10 is further connected to the terminal 200 wiredly or wirelessly, so that the camera module 20 and the terminal 200 can exchange data.

Optionally, the endoscope 100 may further include a probe tube and a cable (not shown). One end of the cable is connected to the endoscope motherboard 10 and an other end of the cable is connected to the camera module 20 to achieve communication between the endoscope motherboard 10 and the camera module 20. The cable may include two sets of cables. One set of the cables is configured to achieve the communication between the endoscope motherboard 10 and the camera module 20. An other set of the cables is configured to achieve control of a flash light of the camera module 20 by the endoscope motherboard 10. The probe tube may be a semi-rigid tube that may be slightly changed in direction. The probe tube is sleeved on the cable to protect the cable.

Optionally, the endoscope 100 may further include a support (not shown). The endoscope motherboard 10 is disposed on the support. A clamping structure may be disposed on the support, to fix the terminal 200 in the bracket.

Specifically, referring to FIG. 2 together, the endoscope motherboard 10 includes a communication interface 11, a wireless communication module 12 and an adapter board 13. The communication interface 11 and the wireless communication module 12 are connected to the adapter board 13. The adapter board 13 is connected to the camera module 20.

The communication interface 11 or the wireless communication module 12 is configured to be connected to the terminal 200 to achieve a wired connection or a wireless connection between the endoscope motherboard 10 and the terminal 200. Priorities may be set for the wired connection and the wireless connection. When the priority of the wired connection is higher, if the terminal 200 is connected to the adapter board by using both the communication interface 11 and the wireless communication module 12, communication between the terminal 200 and the communication interface 11 is preferentially performed. When the priority of the wireless connection is higher, if the terminal 200 is connected to the adapter board by using both the communication interface 11 and the wireless communication module 12, communication between the terminal 200 and the wireless communication module 12 is preferentially performed.

The communication interface 11 may be a universal serial bus (USB) interface. The communication interface 11 may be embedded in the adapter board 13 or the support to facilitate the connection to the adapter board 13. The communication interface 11 is configured to be connected to the terminal 200. Therefore, the terminal 200 is connected to the adapter board 13 by using the communication interface 11. For example, when the terminal 200 is a smart phone, the communication interface 11 and an OTG interface of the terminal 200 may be connected by using an On-the-go (OTG) patch cord. Therefore, the communication interface 11 is connected to the terminal 200. When the communication interface 11 is connected to the terminal 200, the endoscope motherboard 10 is connected to the terminal 200 wiredly.

The wireless communication module 12 may be one or more of a radio frequency signal communication module, an infrared signal communication module and a Wi-Fi signal communication module. The wireless communication module 12 is configured to be connected to the terminal 200 wirelessly.

The adapter board 13 includes a detection module 131 and a gating switch 132. The detection module 131 is connected to the gating switch 132. The gating switch 132 is connected to the communication interface 11 and the wireless communication module 12. The detection module 131 is configured to detect a status of connection between the communication interface 11 or the wireless communication module 12 and the terminal 200 and transmit a signal to the gating switch 132 according to the status of connection. The gating switch 132 is configured to connect the camera module 20 to the communication interface 11 or the wireless communication module 12 according to the signal transmitted by the detection module 131.

The detection module 131 may detect the status of connection between the communication interface 11 or the wireless communication module 12 and the terminal 200 by using hardware or software. For example, the detection module 131 may be a detection circuit. The detection module 131 is connected to the communication interface 11. When the communication interface 11 is connected to the terminal 200, the detection circuit outputs a level signal. When the communication interface 11 is not connected to the terminal 200, the detection circuit outputs another level signal. Therefore, the gating switch 132 selectively connects the camera module 20 to the communication interface 11 or the wireless communication module 12 according to the different level signals. For another example, the detection module 131 may be a processor, a single-chip microcomputer or the like. When the communication interface 11 is connected to the terminal 200, the processor receives a signal transmitted by the terminal 200 and then outputs a signal. When the wireless communication module 12 is connected to the terminal 200, the processor receives a signal transmitted by the wireless communication module 12 or the terminal 200 and then outputs another signal. Therefore, the gating switch 132 selectively connects the camera module 20 to the communication interface 11 or the wireless communication module 12 according to the different signals.

The gating switch 132 may be a switch circuit, an electronic switch, a gate, a selector, a distributor or hardware logic having similar functions. The switch circuit may be implemented by using a hardware chip, a circuit device, a combined circuit or a logic circuit having a gating function. The specific implementation or a combination manner is not limited in the disclosure. Such implementations all fall within the scope of the disclosure. In this embodiment, the gating switch 132 is connected to the communication interface 11, the wireless communication module 12 and the camera module 20. The gating switch 132 is configured to selectively connect the camera module 20 to the communication interface 11 or the wireless communication module 12 according to the signal transmitted by the detection module 131. For example, when receiving a high-level signal transmitted by the detection module 131, the gating switch 132 selectively connects the camera module 20 to the communication interface 11. When receiving a low-level signal transmitted by the detection module 131, the gating switch 132 selectively connects the camera module 20 to the wireless communication module 12.

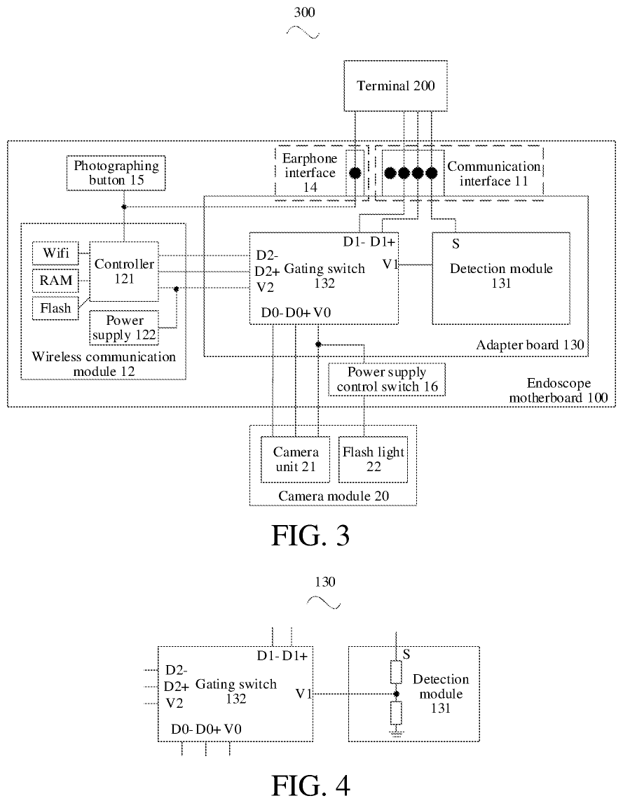

Specifically, referring to FIG. 3, in this embodiment, the detection module 131 is connected to the communication interface 11. When the communication interface 11 is connected to the terminal 200, the detection module 131 is configured to transmit a first signal to the gating switch 132. Therefore, the gating switch 132 connects the camera module 20 to the communication interface 11. When the communication interface 11 is not connected to the terminal 200, the detection module 131 is configured to transmit a second signal to the gating switch 132. Therefore, the gating switch 132 connects the camera module 20 to the wireless communication module 12.

As shown in FIG. 4, the detection module 131 may be a circuit composed of two resistors. One end of one of the resistors is configured to be connected to the communication interface 11. Another end of the resistor is connected to one end of another of the resistors. Another end of the other resistor is grounded. A common connection end of the two resistors is connected to the gating switch 132. When the communication interface 11 is connected to the terminal 200, the terminal 200 outputs a high-level signal to the detection module 131 by using the communication interface 11. Therefore, the detection module 131 receives the high-level signal and transmits a high-level signal, that is, a first signal to the gating switch 132. In this case, the gating switch 132 connects the camera module 20 to the communication interface 11 according to the first signal. When the communication interface 11 is not connected to the terminal 200, a V1 terminal of the gating switch 132 is grounded. That is to say, the detection module 131 transmits a low-level signal, that is, a second signal to the gating switch 132. In this case, the gating switch 132 connects the camera module 20 to the wireless communication module 12 according to the second signal. The detection module 131 adopting two resistors has a simple structure and low costs.

It should be noted that, when the communication interface 11 is not connected to the terminal 200, the terminal 200 communicates with the camera module 20 wirelessly. When the communication interface 11 is connected to the terminal 200, the wireless connection between the terminal 200 and the camera module 20 is disconnected.

Optionally, in some other embodiments, the detection module 131 may be implemented in other forms. When the communication interface 11 is connected to the terminal 200, the detection module 131 is configured to detect whether a signal from the communication interface 11 is received; and if the signal from the communication interface 11 is received, transmit a first signal to the gating switch 132. When the communication interface 11 is not connected to the terminal 200, the detection module 131 configured to detect whether a signal from the wireless communication module 12 is received, and if the signal from the wireless communication module 12 is received, transmit a second signal to the gating switch 132.

Specifically, in this embodiment, the camera module 20 may include a camera unit 21 and a flash light 22. The camera unit 21 and the flash light 22 are connected to the gating switch 132. Specifically, the camera unit 21 is connected to a D0+ terminal, a D0− terminal and a V0 terminal of the gating switch 132, to achieve the connection to the communication interface 11 or the wireless communication module 12. The flash light 22 is connected to the V0 terminal of the gating switch 132, to acquire power from the terminal 200 or the wireless communication module 12.

The camera unit 21 may include a camera lens, a camera, an image capture card and the like. The camera unit 21 is configured to take pictures or record videos. Certainly, in some other embodiments, one or more camera units 21 may be selectively used as required for capturing images, which is not limited herein.

When the terminal 200 performs wired communication, that is, when the communication interface 11 is connected to the terminal 200, a flashlight function of the terminal 200 cannot be used, to reduce power consumption of the terminal 200. In this way, the work time of the endoscope motherboard 10 is prolonged.

In this embodiment, in the endoscope motherboard 10, the communication interface 11 and the wireless communication module 12 are connected to the adapter board 13 and the adapter board 13 is connected to the camera module 20. The adapter board 13 includes the detection module 131 and the gating switch 132. The detection module 131 detects the status of the connection between the communication interface 11 or the wireless communication module 12 and the terminal 200 and transmits the signal to the gating switch 132 according to the status of connection. The gating switch 132 connects the camera module 20 to the communication interface 11 or the wireless communication module 12 according to the signal transmitted by the detection module 131. In this way, the endoscope motherboard 10 can support both wired communication and wireless communication. Therefore, a user may freely select the wired communication or the wireless communication, improving user experience.

In some embodiments, referring to FIG. 3 again, the communication interface 11 may include a signal transmission port and a power supply port.

The signal transmission port includes two signal transmission ports. One ends of the two signal transmission ports are configured to be respectively connected to a D1+ terminal and a D1− terminal of the gating switch 132. Other ends of the two signal transmission ports are configured to be connected to a USB OTG interface of the terminal 200. The signal transmission port is used for data exchange between the terminal 200 and the camera module 20. When the communication interface 11 is connected to the terminal 200, the gating switch 132 connects the signal transmission port to the camera module 20. The signal transmission port transmits, to the terminal 200, an image signal transmitted by the camera module 20 so that the terminal 200 receives the image signal, and/or transmits, to the camera module 20, a control signal transmitted by the terminal 200 so that the camera module 20 performs photographing according to the control signal. For example, referring to FIG. 3 and FIG. 4 together, when the communication interface 11 is connected to the terminal 200, the D1+ terminal and the D1− terminal of the gating switch 132 are respectively connected to the D0+ terminal and the D0− terminal of the gating switch 132. A user triggers a control signal by using software installed in the terminal 200. Therefore, the terminal 200 transmits the control signal to the camera module 20 and the camera module 20 performs photographing according to the control signal. After completing the photographing, the camera module 20 transmits the image signal to the terminal 200 by using the communication interface 11.

The terminal 200 may support a USB video class (UVC) protocol. Therefore, the terminal 200 may receive video data in an MJpeg format transmitted by the camera module 20 to the terminal 200 and perform MJpeg decoding and playback. The terminal 200 may further support an H.264 encoding function. During video recording by the camera module 20, a video stream transmitted to the terminal 200 by the camera module 20 is encoded into an H.264 format for storage.

It should be noted that the image signal in the present application may include picture signals and video signals in various formats. A specific implementation or a combination manner is not limited in the disclosure. Such implementations all fall within the scope of the disclosure.

One end of the power supply port is configured to be connected to an S terminal of the detection module 131. Another end of the power supply port is configured to be connected to the USB OTG interface of the terminal 200. The power supply port is configured to cause the terminal 200 to supply power to the camera module 20. When the communication interface 11 is connected to the terminal 200, the gating switch 132 connects the power supply port to the camera module 20. Therefore, the terminal 200 supplies power to the camera module 20 by using the power supply port. For example, referring to FIG. 3 and FIG. 4 together, when the communication interface 11 is connected to the terminal 200 and a high level signal is inputted to the S terminal of the detection module 131, the detection module 131 transmits a high-level signal (that is, the first signal) to the V1 terminal of the gating switch 132. Therefore, the D1+ terminal, the D1− terminal and the V1 terminal of the gating switch 132 are respectively connected to the D0+ terminal, the D0− terminal and the V0 terminal of the gating switch 132, to transmit the high-level signal to the camera module 20 by using the V0 terminal.

Referring to FIG. 3 again, the wireless communication module 12 may include a signal controller 121 and a power supply 122.

The controller 121 may include a processor, an application specific integrated circuit (ASIC) having control and processing functions, a field programmable gate array (FPGA), a single-chip microcomputer and the like. The controller 121 is connected to a D2+ terminal and a D2− terminal of the gating switch 131 and is used for data exchange between the terminal 200 and the camera module 20. When the wireless communication module 12 is connected to the terminal 200, the controller 121 receives the image signal transmitted by the camera module 20 and wirelessly transmits the image signal to the terminal 200, and/or wirelessly receives the control signal transmitted by the terminal 200. Therefore, the camera module 20 performs photographing according to the control signal. For example, as shown in FIG. 3, when the communication interface 11 is not connected to the terminal 200 and the wireless communication module 12 is connected to the terminal 200, the D2+ terminal and the D2− terminal of the gating switch 132 are respectively connected to the D0+ terminal and the D0− terminal of the gating switch 132. A user triggers a control signal by using software installed in the terminal 200, so that the terminal 200 wirelessly transmits the control signal to the controller 121. The controller 121 wirelessly receives the control signal transmitted by the terminal 200 and transmits the control signal to the camera module 20 by using the D0+ terminal and the D0− terminal. Therefore, the camera module 20 performs photographing according to the control signal. After completing the photographing, the camera module 20 transmits an image signal to the controller 121 by using the D0+ terminal and the D0− terminal. The controller 121 wirelessly transmits the image signal to the terminal 200.

The controller 121 may support the USB video class (UVC) protocol. Therefore, the controller 121 may receive video data in an MJpeg format transmitted by the camera module 20 to the controller 121 and perform MJpeg decoding and playback. The controller 121 may further support an H.264 encoding function. During video recording by the camera module 20, a video stream transmitted to the controller 121 by the camera module 20 is encoded into an H.264 format for storage, and then wirelessly transmitted to the terminal 200.

The power supply 122 is connected to the controller 121 and is configured to supply power to the controller 121. The power supply 122 is further connected to the gating switch 132. Therefore, when the wireless communication module 12 is connected to the terminal 200, the power supply supplies power to the camera module 20. For example, as shown in FIG. 3, when the communication interface 11 is not connected to the terminal 200 and the wireless communication module 12 is connected to the terminal 200, a V2 terminal of the gating switch 132 is connected to the V0 terminal of the gating switch 132. Therefore, the power supply 122 supplies power to the camera module 20 by using the V2 terminal and the V0 terminal.

Optionally, the wireless communication module 12 may further include a memory. The memory is connected to the controller 121 and is configured to store image data and the like. The memory may include a volatile memory, such as a random access memory (RAM), a static random access memory (SRAM), and a double data rate synchronous dynamic random access memory (DDR SDRAM). The memory may also include a non-volatile memory, such as a flash memory, a hard disk drive (HDD), or a solid-state drive (SSD), and an electrically erasable programmable read only memory (EEPROM). The memory may also include a combination of the foregoing types of memories. The memory may be an independent memory, or may be a memory inside a chip (such as a processor chip) or a module having a storage function. The memory may store a computer program (such as a control program for a positioning module, a vehicle diagnosis program, a functional module and the like), a computer instruction, an operating system, data, a database and the like. The memory may be partitioned for storage.

In some embodiments, referring to FIG. 3 again, the endoscope motherboard 10 may further include an earphone interface 14 and a photographing button 15.

One end of the earphone interface 14 is connected to the photographing button 15. Another end is configured to be connected to an earphone interface of the terminal 200. When a wired connection is used, the communication interface 11 is connected to the USB OTG interface of the terminal 200 and the earphone interface 14 is connected to the earphone interface of the terminal 200. The earphone interface 14 is configured to transmit, to the terminal 200, a trigger signal transmitted by the photographing button 15. Therefore, the terminal 200 transmits a control signal according to the trigger signal. For example, when the wired connection is used, a user presses the photographing button 15 for photographing. The photographing button 15 transmits a trigger signal. The trigger signal is transmitted to the terminal 200 by using the earphone interface 14 and the earphone interface of the terminal 200. The terminal 200 receives the trigger signal. In this case, the terminal 200 transmits a control signal to control the camera module 20 to perform photographing.

The photographing button 15 is further connected to the controller 121. When the wireless communication module 12 is connected to the terminal 200, the controller 121 is configured to receive the trigger signal transmitted by the photographing button 15 and wirelessly transmit the trigger signal to the terminal 200. Therefore, the terminal 200 transmits the control signal according to the trigger signal. For example, when the wireless connection is used, the wireless communication module 12 is connected to the terminal 200, and the earphone interface 14 is not connected to the terminal 200. A user presses the photographing button 15 for photographing. The photographing button 15 transmits a trigger signal to the controller 121. The controller 121 wirelessly transmits the trigger signal to the terminal 200. The terminal 200 receives the trigger signal. The terminal 200 returns a control signal to the controller 121. An application of the terminal 200 updates control information synchronously. The controller 121 controls, according to control signal, the camera module 20 to perform photographing.

In some embodiments, referring to FIG. 3 again, the endoscope motherboard 10 may further include a power supply control switch 16. One end of the power supply control switch 16 is connected to the V0 terminal of the gating switch 132. Another end of the power supply control switch 16 is connected to the flash light 22 of the camera module 20 to control a brightness of the flash light 22. Optionally, the power supply control switch 16 may be a sliding rheostat.

In this embodiment, in the endoscope motherboard 10, the communication interface 11 and the wireless communication module 12 are connected to the adapter board 13 and the adapter board 13 is connected to the camera module 20. The adapter board 13 includes the detection module 131 and the gating switch 132. The detection module 131 detects the status of the connection between the communication interface 11 or the wireless communication module 12 and the terminal 200 and transmits the signal to the gating switch 132 according to the status of connection. The gating switch 132 connects the camera module 20 to the communication interface 11 or the wireless communication module 12 according to the signal transmitted by the detection module 131. In this way, the endoscope motherboard 10 can support both wired communication and wireless communication. Therefore, a user may freely select the wired communication or the wireless communication, improving user experience.

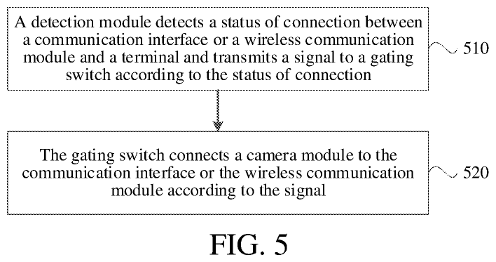

Referring to FIG. 5, FIG. 5 is a schematic flowchart of a detection method according to an embodiment of the disclosure. As shown in FIG. 5, the detection method is applicable to the endoscope motherboard 10 in the foregoing embodiments. The method includes the following steps:

510. The detection module detects a status of connection between the communication interface or the wireless communication module and the terminal and transmitting a signal to the gating switch according to the status of connection.

In this embodiment, when detecting that the communication interface 11 has been connected to the terminal 200, the detection module 131 of the endoscope motherboard 10 transmits a first signal to the gating switch 132. When detecting that the communication interface 11 is not connected to the terminal 200, the detection module 131 of the endoscope motherboard 10 transmits a second signal to the gating switch 132. For example, as shown in FIG. 4, when the communication interface 11 is connected to the terminal 200, a high level signal is inputted to an S terminal of the detection module 131. The detection module 131 transmits a high-level signal to the V1 terminal of the gating switch 132. When the communication interface 11 is not connected to the terminal 200, the V1 terminal of the gating switch 132 is grounded. That is to say, the detection module 131 transmits a low-level signal to the V1 terminal of the gating switch 132.

Optionally, in some other embodiments, the detection module 131 may be implemented by other circuits or modules. The detection module 131 may further detect the status of connection between the communication interface 11 or the wireless communication module 12 and the terminal 200 in the following manner: When detecting that the communication interface 11 is already connected to the terminal 200, the detection module 131 of the endoscope motherboard 10 transmits a first signal to the gating switch 132. Alternatively, when receiving a signal from the wireless communication module 12, the detection module 131 of the endoscope motherboard 10 transmits a second signal to the gating switch 132.

520: The gating switch connects the camera module to the communication interface or the wireless communication module according to the signal.

In this embodiment, if receiving the first signal, the gating switch 132 connects the camera module 20 to the communication interface 11. Alternatively, if receiving the second signal, the gating switch 132 connects the camera module 20 to the wireless communication module 12. For example, as shown in FIG. 4, when receiving the high-level signal, the gating switch 132 connects D1+ to D0+, D1− to D0− and V1 to V0, thereby connecting the communication interface 11 to the camera module 20. Alternatively, when receiving the low-level signal, the gating switch 132 connects D2+ to D0+, D2− to D0− and V2 to V0, thereby connecting the wireless communication module 12 to the camera module 20.

In this embodiment, according to the detection method, the detection module 131 detects the status of connection between the communication interface 11 or the wireless communication module 12 and the terminal 200 and transmits the signal to the gating switch 132 according to the status of connection. The gating switch 132 connects the camera module 20 to the communication interface 11 or the wireless communication module 12 according to the signal transmitted by the detection module 131. In this way, the endoscope motherboard 10 can support both wired communication and wireless communication. Therefore, a user may freely select the wired communication or the wireless communication, improving user experience.

It should be noted that the described apparatus embodiment is merely exemplary. The units described as separate parts may or may not be physically separate. Parts displayed as units may or may not be physical units, may be located in one position, or may be distributed on multiple network units. Some or all of the modules may be selected according to actual needs to implement the objectives of the solutions of the embodiments.

Through the description of the foregoing embodiments, those of ordinary skill in the art may clearly understand that the embodiments may be implemented by software in combination with a universal hardware platform, and may certainly be implemented by hardware. Those of ordinary skill in the art may understand that all or some of the processes of the methods in the embodiments may be implemented by a computer program instructing relevant hardware. The program may be stored in a computer-readable storage medium. During execution of the program, the processes of the method embodiments may be performed. The storage medium may be a magnetic disk, an optical disc, a read-only memory (ROM), a RAM or the like.

It should be noted that the foregoing embodiments are merely used for describing the technical solutions of the disclosure, but are not intended to limit the disclosure. Under the concept of the disclosure, the technical features in the foregoing embodiments or different embodiments may be combined, the steps may be implemented in any sequence, and there may be many other changes in different aspects of the disclosure as described above. For brevity, those are not provided in detail. Although the disclosure is described in detail with reference to the foregoing embodiments, those of ordinary skill in the art should understand that, they may still make modifications to the technical solutions described in the foregoing embodiments or make equivalent replacements to some technical features thereof, without departing from the scope of the technical solutions of the embodiments of the disclosure.

What is claimed is:

1. An endoscope motherboard, configured to be connected to a terminal and a camera module, the endoscope motherboard comprising a communication interface, a wireless communication module and an adapter board, the communication interface and the wireless communication module connected to the adapter board, and the adapter board connected to the camera module;
the communication interface or the wireless communication module configured to be connected to the terminal;
the adapter board comprising a detection module and a gating switch;
the detection module configured to detect a status of connection between the communication interface and the terminal or a status of connection between the wireless communication module and the terminal, and transmit a signal to the gating switch according to the status of connection; and
the gating switch configured to connect the camera module to the communication interface or the wireless communication module according to the signal.

2. The endoscope motherboard according to claim 1, wherein the communication interface is connected to the detection module and the gating switch; and
when the communication interface is connected to the terminal, the detection module is configured to transmit a first signal to the gating switch, so that the gating switch connects the camera module to the communication interface; or
when the communication interface is not connected to the terminal, the detection module is configured to transmit a second signal to the gating switch, so that the gating switch connects the camera module to the wireless communication module.

3. The endoscope motherboard according to claim 2, wherein
when the communication interface is not connected to the terminal, the detection module is configured to detect whether a signal from the wireless communication module is received; and
if the signal from the wireless communication module is received, transmit the second signal to the gating switch.

4. The endoscope motherboard according to claim 2, wherein the communication interface comprises a signal transmission port;
when the communication interface is connected to the terminal, the signal transmission port configured to transmit an image signal to the terminal, and/or transmit a control signal to the camera module, so that the camera module performs photographing according to the control signal, wherein the image signal is transmitted by the camera module and the control signal is transmitted by the terminal.

5. The endoscope motherboard according to claim 4, wherein the communication interface further comprises a power supply port;
when the communication interface is connected to the terminal, the terminal is configured to supply power to the camera module through the power supply port.

6. The endoscope motherboard according to claim 4, wherein the endoscope motherboard further comprises an earphone interface and a photographing button connected to the earphone interface;
the earphone interface configured to be connected to the terminal, and transmit a trigger signal to the terminal, so that the terminal transmits the control signal according to the trigger signal, wherein the trigger signal is transmitted by the photographing button.

7. The endoscope motherboard according to claim 6, wherein the wireless communication module comprises a controller connected to the gating switch;
when the wireless communication module is connected to the terminal, the controller is configured to receive the image signal transmitted by the camera module, and wirelessly transmit the image signal to the terminal and/or wirelessly receive the control signal transmitted by the terminal, so that the camera module performs photographing according to the control signal.

8. The endoscope motherboard according to claim 7, wherein the photographing button is further connected to the controller; and
when the wireless communication module is connected to the terminal, the controller is further configured to receive the trigger signal transmitted by the photographing button and wirelessly transmit the trigger signal to the terminal, so that the terminal transmits the control signal according to the trigger signal.

9. The endoscope motherboard according to claim 8, wherein the wireless communication module further comprises a power supply connected to the gating switch;
when the wireless communication module is connected to the terminal, the power supply is configured to supply power to the camera module.

10. The endoscope motherboard according to claim 1, further comprising a power supply control switch;
one end of the power supply control switch being connected to the gating switch, and another end of the power supply control switch being connected to a flash light of the camera module to control a brightness of the flash light of the camera module.

11. The endoscope motherboard according to claim 1, wherein the wireless communication module is one or more of a radio frequency signal communication module, an infrared signal communication module and a Wi-Fi signal communication module.

12. An endoscope, comprising a camera module and an endoscope motherboard, the endoscope motherboard configured to be connected to a terminal and a camera module, the endoscope motherboard comprising a communication interface, a wireless communication module and an adapter board, the communication interface and the wireless communication module connected to the adapter board, and the adapter board connected to the camera module;
the communication interface or the wireless communication module configured to be connected to the terminal;
the adapter board comprising a detection module and a gating switch;
the detection module configured to detect a status of connection between the communication interface and the terminal or a status of connection between the wireless communication module and the terminal, and transmit a signal to the gating switch according to the status of connection; and
the gating switch configured to connect the camera module to the communication interface or the wireless communication module according to the signal.

13. The endoscope according to claim 12, wherein the communication interface is connected to the detection module and the gating switch; and
when the communication interface is connected to the terminal, the detection module is configured to transmit a first signal to the gating switch, so that the gating switch connects the camera module to the communication interface; or
when the communication interface is not connected to the terminal, the detection module is configured to transmit a second signal to the gating switch, so that the gating switch connects the camera module to the wireless communication module.

14. The endoscope according to claim 13, wherein
when the communication interface is not connected to the terminal, the detection module is configured to detect whether a signal from the wireless communication module is received; and
if the signal from the wireless communication module is received, transmit the second signal to the gating switch.

15. The endoscope according to claim 13, wherein the communication interface comprises a signal transmission port;
when the communication interface is connected to the terminal, the signal transmission port configured to transmit an image signal to the terminal, and/or transmit a control signal to the camera module, so that the camera module performs photographing according to the control signal, wherein the image signal is transmitted by the camera module and the control signal is transmitted by the terminal.

16. The endoscope according to claim 15, wherein the communication interface further comprises a power supply port;
when the communication interface is connected to the terminal, the terminal is configured to supply power to the camera module through the power supply port.

17. The endoscope according to claim 15, wherein the endoscope motherboard further comprises an earphone interface and a photographing button connected to the earphone interface;
the earphone interface configured to be connected to the terminal, and transmit a trigger signal to the terminal, so that the terminal transmits the control signal according to the trigger signal, wherein the trigger signal is transmitted by the photographing button.

18. The endoscope according to claim 17, wherein the wireless communication module comprises a controller connected to the gating switch;
when the wireless communication module is connected to the terminal, the controller is configured to receive the image signal transmitted by the camera module, and wirelessly transmit the image signal to the terminal and/or wirelessly receive the control signal transmitted by the terminal, so that the camera module performs photographing according to the control signal.

19. The endoscope according to claim 12, wherein the endoscope motherboard further comprises a power supply control switch, one end of the power supply control switch being connected to the gating switch, and another end of the power supply control switch being connected to a flash light of the camera module to control a brightness of the flash light of the camera module.

20. A detection method, applicable to an endoscope motherboard, the endoscope motherboard comprising a communication interface, a wireless communication module and an adapter board, the communication interface and the wireless communication module connected to the adapter board, and the adapter board being connected to the camera module;
the communication interface or the wireless communication module configured to be connected to the terminal;
the adapter board comprising a detection module and a gating switch;
the method comprising:
detecting, by the detection module, a status of connection between the communication interface and the terminal or a status of connection between the wireless communication module and the terminal, and transmitting a signal to the gating switch according to the status of connection; and
connecting, by the gating switch, the camera module to the communication interface or the wireless communication module according to the signal.
